(19) United States
(12) Patent Application Publication
(10) Pub. No.: US 2026/0124357 A1
(43) Pub. Date: May 7, 2026

(54) PREFILLED RAPID BOLUS SYRINGE

(71) Applicant: CareFusion 303, Inc., San Diego, CA (US)

(72) Inventors: Ryan Callahan, Long Beach, CA (US); Siddarth K. Shevgoor, Mission Viejo, CA (US); Gene E. Mason, La Habra Heights, CA (US); Likhith Nalluri, Brea, CA (US)

(21) Appl. No.: 18/936,620

(22) Filed: Nov. 4, 2024

(51) Int. Cl. A61M 5/145; A61M 5/168

(52) U.S. Cl. CPC A61M 5/14526; A61M 5/1456; A61M 5/168; A61M 2005/14506

(57) ABSTRACT

A syringe may be pre-filled with fluid and connected to a rapid infusion attachment to deliver a rapid bolus of fluid to a patient. The rapid infusion attachment may comprise a spring that, when compressed, provides a spring force to the plunger of the syringe. The device comprising the syringe and the rapid infusion attachment may be shipped and stored such that the syringe is pre-filled and the spring is pre-loaded, or compressed. The device may also be shipped and stored with the spring uncompressed, and a clinician or other user may compress the spring prior to attaching the device to an IV set or other fluid pathway. The device may further comprise a mechanism that allow the clinician to control the fluid flow. The device may also further comprise a locking mechanism that can be released when the fluid in the syringe is to be injected.

### FIELD OF THE INVENTION

The present disclosure generally relates to syringes, and, in particular, to rapid fluid delivery.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. In cases of trauma with large loss of blood or sepsis, the initial response from healthcare providers is often to provide rapid fluid resuscitation in order to restore blood pressure. This is often achieved by a gravity IV set.

### SUMMARY

Gravity sets can take significant time to set up, and gravity flow rate is often insufficient. Dedicated rapid infusers are not portable and are only available in high end medical centers.

One or more embodiments of the present disclosure are directed to a device for rapid fluid delivery comprising a pre-filled syringe for providing an infusion, the syringe comprising a syringe barrel for holding a fluid, and a syringe plunger connected to a proximal end of the barrel, and a rapid infusion attachment, wherein the rapid infusion attachment provides increased pressure to drive an infusion, the rapid infusion attachment comprising an attachment plunger connected to the syringe plunger by a spring, and a housing that sheaths a portion of the attachment plunger, and an activation mechanism that controls flow of the fluid from the syringe.

In some embodiments, the attachment plunger comprises a base portion and a hollow channel portion, wherein the hollow channel portion houses the spring.

In some embodiments, the attachment plunger slides to sheath the hollow channel portion within the housing.

In some embodiments, the spring is attached to a proximal end of the syringe plunger and the base portion of the hollow channel of the attachment plunger, and when the hollow channel portion is sheathed within the housing, the spring is in a compressed position, and the spring provides an injection force to the syringe plunger when the spring is in the compressed position.

In some embodiments, the hollow channel portion is sheathed within the housing, compressing the spring, before being packaged.

In some embodiments, the hollow channel portion of the attachment plunger comprises exterior threads and the housing comprises interior threads that interact with the exterior threads of the hollow channel portion, and the attachment plunger twists to sheath the hollow channel portion within the housing.

In some embodiments, the attachment plunger comprises a protruding flange at a distal end of the hollow channel portion and the housing comprises interior grooves that catch the protruding flange of the attachment plunger such that when a compression force is applied to a proximal end of the attachment plunger, the hollow channel portion is sheathed within the housing and the protruding flange interacts with the interior grooves to keep the attachment plunger in its sheathed position.

In some embodiments, the activation mechanism is attached to a needle end of the syringe and the activation mechanism stops the fluid from flowing out of the syringe when closed and allows the fluid to flow out of the syringe when open.

In some embodiments, a distal end of the syringe facilitates attachment of the device to a venous access pathway.

In some embodiments, the syringe barrel is pre-filled with sterile saline or other fluid for a rapid fluid injection.

In some embodiments, the attachment housing removably clips to the pre-filled syringe.

One or more embodiments of the present disclosure are directed to a device for rapid fluid delivery, comprising a pre-filled syringe for providing an infusion, where the syringe comprises a syringe barrel with a proximal end and a distal end, wherein the barrel is filled with a fluid, and a syringe plunger with a gasket end and a free end, wherein the gasket end is located within the barrel, and a device plunger with a base portion and an elongate core portion, wherein the elongate core portion comprises a hollow chamber configured to receive the syringe plunger, a housing with a proximal end and a distal end, wherein the distal end of the housing is attached to the proximal end of the syringe barrel, wherein the housing is configured to receive the elongate core portion of the device plunger, and a pre-compressed spring attached to the free end of the syringe plunger and to the device plunger, wherein the pre-compressed spring provides an injection force to the syringe plunger.

In some embodiments, the device further comprises a flow regulating device attached to a tip of the syringe barrel, wherein the flow regulating device controls fluid flow from the syringe barrel.

In some embodiments, the flow regulating device comprises a valve mechanism configured to open and close a fluid pathway.

In some embodiments, opening the fluid pathway permits the fluid to be pushed out of the syringe barrel as the spring decompresses.

In some embodiments, the valve mechanism is electronic and programmable such that the valve mechanism opens and closes the fluid pathway on a timer to administer the fluid at a predetermined rate.

In some embodiments, the pre-filled syringe is configured to be inserted into an IV system or a patient.

One or more embodiments of the present disclosure are directed to a device for automatic syringe dispensing that comprises a syringe barrel having a tip end and a base end, a spring located within the syringe barrel attached to the base end of the syringe barrel, a locking mechanism located within the syringe barrel and adjacent to the spring, and a plunger located within the syringe barrel and adjacent to the locking mechanism, wherein the locking mechanism prohibits movement of the plunger when active and permits movement of the plunger when released.

In some embodiments, the syringe barrel is filled with a fluid, and the fluid is pushed out of the syringe barrel by the spring when the locking mechanism is released.

In some embodiments, the device further comprises a flow regulator attached to the tip end of the syringe, and the flow regulator is configurable to infuse the fluid at a determined rate.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1A shows an embodiment of a syringe with a rapid fluid attachment.

FIG. 1B shows a cross-section of an embodiment of a syringe with a rapid fluid attachment.

FIG. 2A shows an embodiment of a syringe with a rapid fluid attachment.

FIG. 2B shows a cross-section of an embodiment of a syringe with a rapid fluid attachment.

FIG. 3A shows an embodiment of a syringe with a rapid fluid attachment.

FIG. 3B shows a cross-section of an embodiment of a syringe with a rapid fluid attachment.

FIGS. 4A-4C show an embodiment of a locking mechanism.

FIGS. 5A-5F show embodiments of a flow regulating device in various perspective views.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of fluid connectors, such embodiments can be used in other systems that benefit from creating sterile connections. Furthermore, various applications of such embodiments and modifications thereto, which may occur to those who are skilled in the art, are also encompassed by the general concepts described herein.

The disclosed syringe overcomes several challenges discovered with respect to rapid fluid delivery. In cases of trauma with large loss of blood or sepsis, the initial response from healthcare providers is often to provide rapid fluid resuscitation in order to restore blood pressure. This is typically achieved with a gravity IV set, often paired with a pressure cuff. In Level 1 trauma centers, a dedicated rapid infuser may be used. Gravity sets take significant time to set up, as a bag must be spiked, and the set must be primed. Gravity flow rate is often insufficient and adding a pressure cuff can increase set up time. Dedicated rapid infusers are not portable and are only available in high end medical centers.

Therefore, in accordance with the present disclosure, it is advantageous to provide syringes as described herein that allow for the delivery of a rapid fluid bolus without requiring expensive additional medical equipment and that may be faster to set up than traditional IV sets and pressure bags for rapid fluid resuscitation, and that may be more portable and affordable than dedicated rapid infusion systems. The disclosed syringe includes a device comprising a pre-filled syringe attached to a rapid infusion attachment.

In embodiments of the present disclosure, a pre-filled sterile saline syringe is pre-attached to a rapid infusion attachment that provides increased pressure to drive the infusion. This device allows for a rapid bolus of fluid to be administered in the field with minimal set up time. Additionally, the entire system may be disposable.

In some embodiments, a sterile syringe is pre-filled with saline, dextrose, Ringer's solution, or other fluid. The sterile syringe may hold various syringe volumes. The syringe may be attached to a rapid infusion attachment. The rapid infusion attachment may comprise a spring that can provide an injection force to the plunger of the syringe.

In some embodiments, the device comes pre-filled with sterile saline or other fluid and pre-charged (with the spring already compressed). The pre-filled, pre-charged device may be shipped and stored sealed in a pouch or other packaging. A clinician may be able to open the pouch and attach the syringe to a venous access pathway, such as a catheter, IV extension set, or other pathway. The clinician then may be able to activate the device, delivering a rapid fluid bolus.

In some embodiments, the device comes pre-filed, but not charged (with the spring uncompressed). The clinician may be able to charge the device (compress the spring) before or after attaching the device to a venous access pathway. The clinician may be able to compress the spring by twisting or compressing together different pieces of the rapid fluid attachment. The clinician may then activate the device, delivering a rapid fluid bolus.

In some embodiments, the rapid infusion attachment has a screw design, where a plunger of the attachment can be screwed into a housing section of the attachment. The spring may be housed within the plunger and/or the housing such that twisting the plunger into the housing compresses the spring. The compressed spring may provide a spring force on a plunger of the filled syringe.

In some embodiments, the rapid infusion attachment has a ratcheting design, where a plunger of the attachment has a pawl mechanism that interacts with a grooved surface of a housing section of the attachment. Pushing or otherwise compressing the attachment plunger into the housing section may cause the pawl mechanism to lock in any forward travel of the attachment plunger. The spring may be housed within the plunger and/or the housing such that compressing the plunger into the housing compresses the spring. The compressed spring may provide a spring force on a plunger of the filled syringe.

In some embodiments, the device further comprises an activation mechanism located at the tip end of the syringe. The activation mechanism may be pre-attached to the device such that the device may be shipped and stored with the activation mechanism attached. The activation mechanism may act as an on/off stopcock and may allow a user or clinician to control flow from the syringe. The activation mechanism may be similar to pre-existing control devices that a clinician would be familiar with, leveraging existing knowledge and experience in the field.

In some embodiments, the rapid infusion attachment applies a force to the syringe plunger with the help of the compressed spring. The spring force may create enough pressure to inject the fluid of the syringe into the IV system or a patient. This system may provide a quick and reliable infusion of fluid.

In some embodiments, the system further comprises a flow-regulating device to infuse the fluid or drug of the syringe at a constant flow rate. If a drug needs to be infused at a specific rate, the flow regulator can be set to accomplish this, as the spring force will continue to push the fluid out of the syringe. In emergency scenarios, the syringe may be connected to the IV system and the flow-regulating device may be engaged such that the fluid will be forced into the system with no need for a human to push the fluid.

In some embodiments, where the device also comprises the flow-regulating device, the device may be used as a pump. With the addition of an electronic clamp or gate attachment, the device may be used as a valve. A desired flow rate may be achieved, and a drug may be administered at certain intervals. In some embodiments, the flow-regulating device may comprise an adjustable valve wherein rotating a portion of the flow-regulating device may adjust the device flow rate by adjusting an aperture size of the fluid pathway.

In some embodiments, a locking mechanism is used to hold the syringe plunger in place. With the locking mechanism, the device spring may be loaded or compressed, and the locking mechanism may hold the syringe plunger in place until the mechanism is disengaged. In some embodiments, the locking mechanism includes a pin that holds the syringe in place when the pin is in place and releases the syringe when the pin is removed. In some embodiments, the mechanism may be released by the press of a button, flip of a switch, release of a latch, or other release method.

In some embodiments, different springs with different spring constants are used to achieve a higher or lower spring force. In some embodiments, an electronic valve mechanism can be used and programmed to open and close the fluid pathway. With such an electronic valve mechanism, a timer could be set to administer the drug periodically at desired times. With the help of the flow-regulating device, the drug may be infused at a constant speed or flow rate.

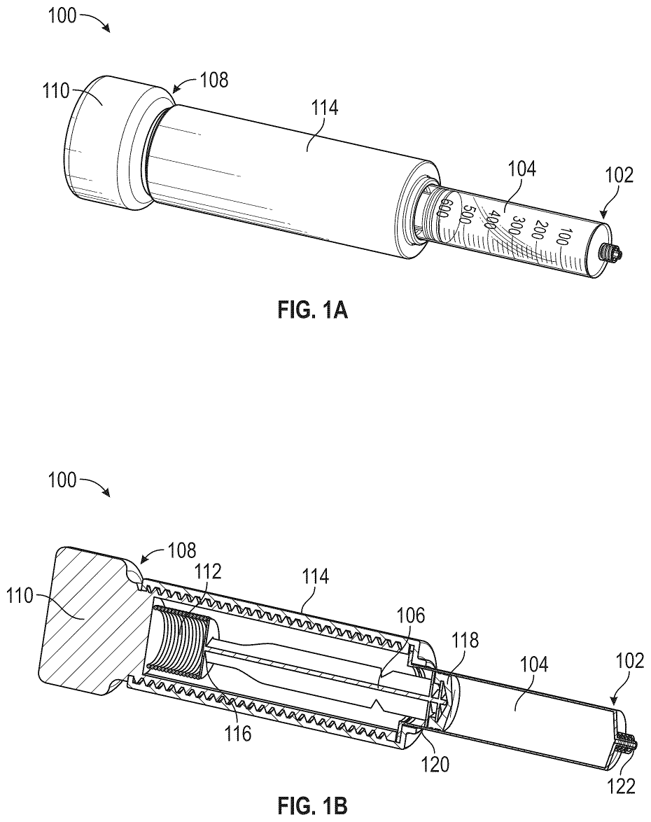

FIG. 1A shows an exemplary device 100 that includes a syringe 102 and an attachment 108. The syringe 102 has a syringe barrel 104, and attachment 108 has an attachment plunger 110 and an attachment housing 114.

FIG. 1B shows an exemplary cross-section of a device 100, such as shown in FIG. 1A. The syringe 102 comprises the syringe barrel 104 and a syringe plunger 106 with a syringe plunger base 116 and a syringe plunger gasket 118. The syringe barrel 104 has a syringe barrel base 120 and a syringe barrel tip 122 The attachment 108 comprises the attachment plunger 110, a spring 112, and the attachment housing 114.

In some embodiments, the syringe is pre-filled with sterile saline or other fluid that can be injected into a patient to raise the patient's blood pressure. In some embodiments, the attachment plunger 110 can be sheathed within the housing 114. The spring 112 may be attached to the plunger base 116 such that when the spring is compressed, the spring exerts a spring force on the plunger 106. Like a traditional syringe pushed by a clinician, the spring force exerted on the syringe plunger base 116 may push the fluid from the syringe barrel 104 out through the syringe barrel tip 122.

In some embodiments, the device 100 is shipped and stored with the spring 112 in a compressed position, such as seen in FIG. 1B. The syringe barrel may also be pre-filled with fluid. Therefore, a clinician may open the device from any packaging that it is stored in, and simply attach the device 100 to an IV set or other fluid pathway and activate the device, and then the bolus of fluid stored in the syringe barrel may be rapidly injected.

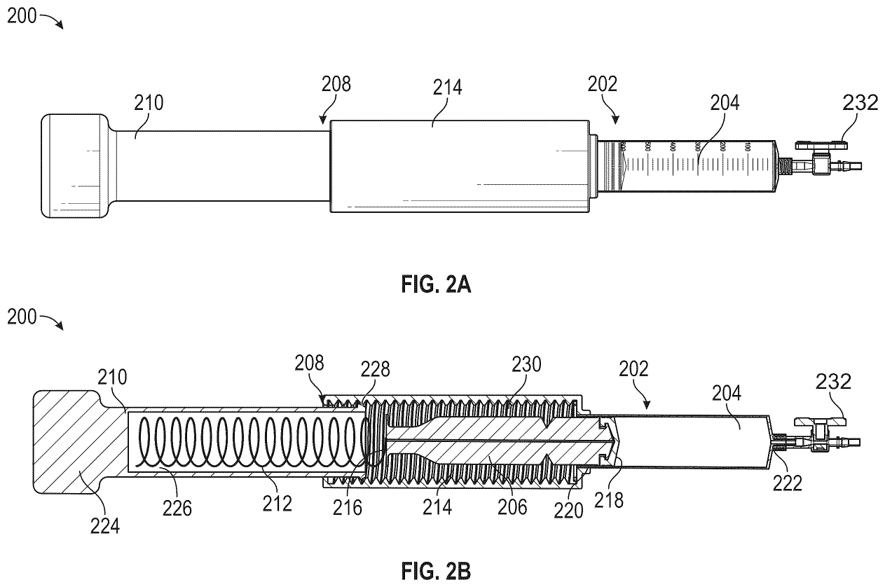

FIG. 2A shows an exemplary device 200. The device 200 includes a syringe 202 and an attachment 208. The syringe 202 comprises syringe barrel 204. The attachment 208 comprises an attachment plunger 210 and an attachment housing 214. An activation mechanism 232 is attached to the tip of the syringe 202.

FIG. 2B shows an exemplary cross-section of the device 200 of FIG. 2A. The syringe 202 comprises the syringe barrel 204 and the syringe plunger 206. The syringe plunger 206 comprises a syringe plunger base 216 and a syringe gasket 218. The syringe barrel 204 comprises a syringe barrel base 220 and a syringe barrel tip 222. The attachment 208 comprises the attachment plunger 210, a spring 212, and the attachment housing 214. The attachment plunger 210 comprises an attachment plunger base 224. The attachment plunger 210 comprises a hollow channel 226 and a lip 228. The attachment housing 214 comprises interior ribs 230.

In some embodiments, the attachment plunger 210 may be pushed into the attachment housing 214, sheathing the plunger in the housing. The attachment plunger 210 and the housing 214 may comprise a pawl system such that the attachment plunger 210 may comprise a lip 228 that extends outwardly from the circumference of the attachment plunger 210, creating a protrusion that interacts with the interior ribs 230 of the housing. The lip 228 may catch onto the ribs 230, trapping the forward motion, or compression into the housing, of the attachment plunger 210. Compressing the attachment plunger 210 into the housing 214 may compress the spring 212, housed within the hollow channel portion 226 of the attachment plunger 210. The lip 228 may catch onto the ribs 230 and hold the attachment plunger 210 in place, counteracting any spring force generated by the compressed spring 212.

In some embodiments, the device 200 further comprises the activation mechanism 232, which is attached to the syringe barrel tip 222. The activation mechanism 232 may allow a clinician or other user of the device 200 to activate the device or allow fluid to flow from the syringe tip. The activation mechanism may allow the device 200 to be charged, with the spring 212 compressed, and keep the fluid from being expelled from the syringe until the clinician or user wants the fluid to be injected. The activation mechanism 232 may, in some embodiments, comprise a handle that may be twisted to adjust the aperture size of the fluid pathway of the syringe barrel tip 222. Opening or activating the activation mechanism 232 may allow fluid to flow from the syringe 202.

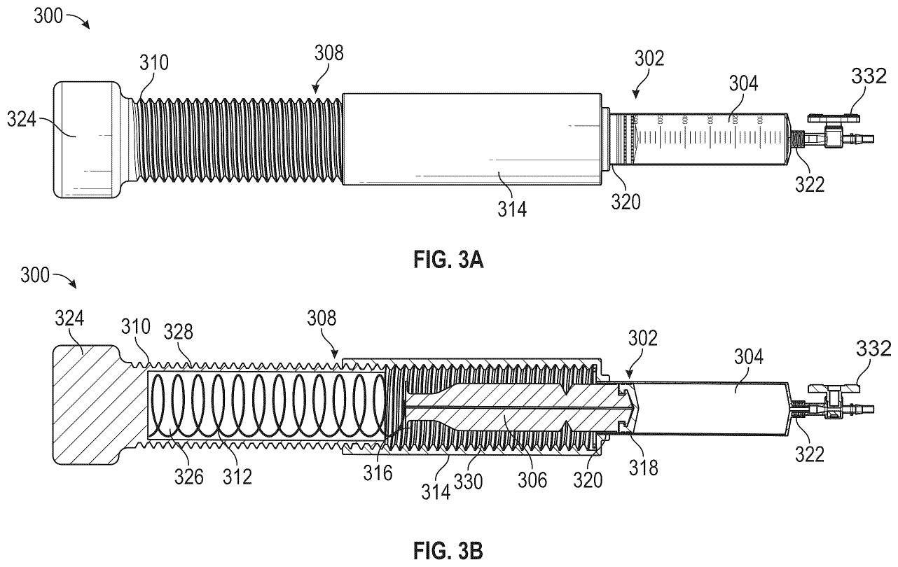

FIG. 3A shows an exemplary device 300. The device 300 includes a syringe 302 and an attachment 308. The syringe 302 comprises syringe barrel 304. The attachment 308 comprises an attachment plunger 310 and an attachment housing 314. An activation mechanism 332 is attached to the tip of the syringe 302.

FIG. 3B shows an exemplary cross-section of the device 300 of FIG. 3A. The syringe 302 comprises the syringe barrel 304 and the syringe plunger 306. The syringe plunger 306 comprises a syringe plunger base 316 and a syringe gasket 318. The syringe barrel 304 comprises a syringe barrel base 320 and a syringe barrel tip 322. The attachment 308 comprises the attachment plunger 310, a spring 312, and the attachment housing 314. The attachment plunger 310 comprises an attachment plunger base 324. The attachment plunger 310 comprises a hollow channel 326 and channel threads 328. The attachment housing 314 comprises interior threads 330.

In some embodiments, the attachment plunger 310 may be twisted into the attachment housing 314, sheathing the plunger in the housing. The attachment plunger 310 and the housing 314 may comprise a threaded system such that the attachment plunger 310 may comprise channel threads 328 that exist along the exterior surface of the attachment plunger 310, creating threads that interacts with the interior threads 330 of the housing. The channel threads 328 may interact with the interior ribs 330, trapping the forward motion, or compression into the housing, of the attachment plunger 210. Twisting the attachment plunger 310 into the housing 314 may compress the spring 312, housed within the hollow channel portion 326 of the attachment plunger 310. The channel threads 338 may interact with the interior threads 330 and hold the attachment plunger 310 in place, counteracting any spring force generated by the compressed spring 312.

In some embodiments, the device 300 further comprises the activation mechanism 332, which is attached to the syringe barrel tip 322. The activation mechanism 332 may allow a clinician or other user of the device 300 to activate the device or allow fluid to flow from the syringe tip. The activation mechanism may allow the device 300 to be charged, with the spring 312 compressed, and keep the fluid from being expelled from the syringe until the clinician or user wants the fluid to be injected. The activation mechanism 332 may, in some embodiments, comprise a handle that may be twisted to adjust the aperture size of the fluid pathway of the syringe barrel tip 322. Opening or activating the activation mechanism 332 may allow fluid to flow from the syringe 302.

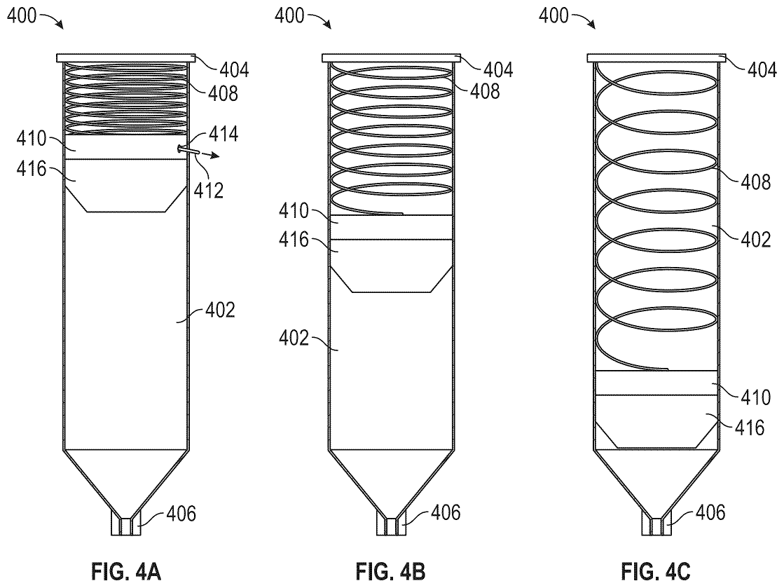

FIGS. 4A-4C show an exemplary pin locking system 400. The pin locking system 400 comprises a syringe barrel 402 that has a base end 404 and a tip end 406. Inside the syringe barrel 402 is a spring 408 and a locking mechanism 410. The locking mechanism 410 has a pin 412 and a pin hole 414. Attached to the locking mechanism 410 is a gasket 416.

In some embodiments, the locking mechanism 410 is attached to the syringe gasket 416 and the spring 408 such that when the locking mechanism 410 is locked, the compressed spring 408 is held in place, and there is no force pushing the fluid from the syringe barrel 402. In some embodiments, such as shown in FIGS. 4A-4C, the locking mechanism 410 includes a pin that holds the mechanism in place when the pin is in place and releases the mechanism when the pin is removed. In some embodiments, other simple locking mechanisms and release methods are used, such as a button or a switch. When the pin 412 is removed from the hole 414, the locking mechanism 410 is released. When the locking mechanism 410 is released, the spring force of the compressed spring 408 pushes the syringe gasket 416 away down the length of the syringe barrel 402 as the spring expands, shown in FIG. 4B and FIG. 4C, pushing fluid out of the syringe. In some embodiments, the pin locking system 400 is connected to a plunger of the syringe. The plunger may be used to help draw fluid into the syringe barrel 402 and compress the spring 408, and then the locking mechanism 410 may be used to hold the spring 408 compressed until the fluid is to be injected.

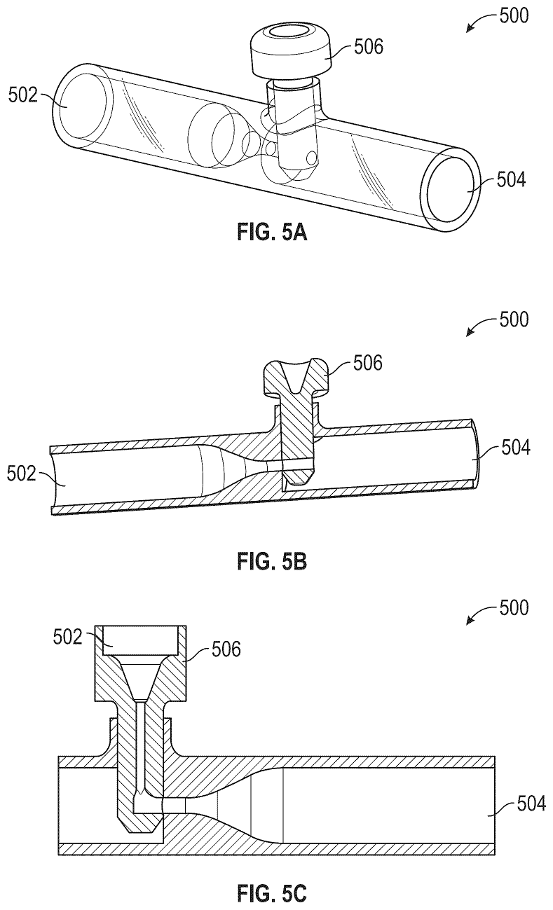

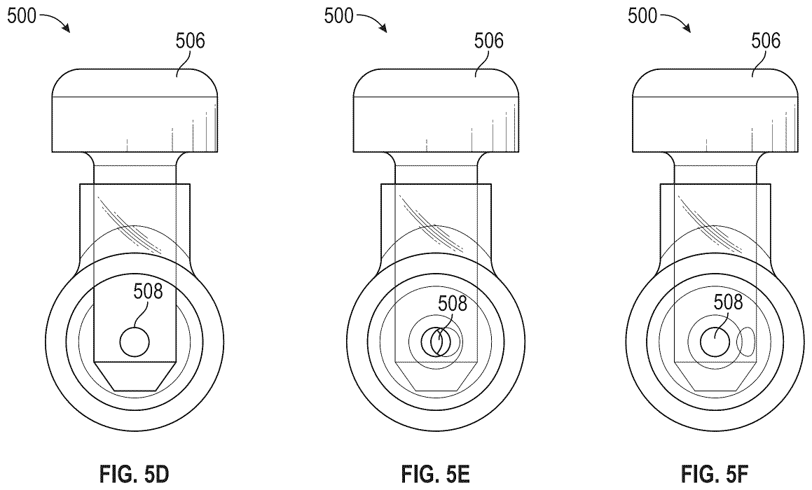

FIGS. 5A-5F show an exemplary flow regulating device 500. The device 500 may have an inflow 502, an outflow 504, and a twist cap 506. Twisting the cap 506 adjusts the width of a valve 508.

In some embodiments, the flow regulating device 500 may be attached to the tip end of a syringe and may be used to control the flow of fluid from the syringe. When used in conjunction with a rapid infusion attachment, as discussed above, a spring force from a compressed spring may push the fluid from a filled syringe. The flow regulating device 500, when attached to the fluid pathway of the syringe, may regulate an aperture of the fluid pathway. Turning the cap 506 may adjust the aperture size of the fluid pathway, affecting the flow rate of the fluid out of the syringe. The flow regulating device 500 may be set such that the fluid pathway is completely blocked, stopping any fluid flow from the syringe. Turning open or activating the flow regulating device may allow fluid to flow from the syringe. When used in conjunction with the rapid infusion attachment, the fluid may be pushed by the expanding spring.

In some embodiments, the inflow 502 is connected to the tip end of a syringe, and the outflow 504 is connected to an IV set or other fluid pathway connecting to a patient. Therefore, the flow regulating device 500 may be used to regulate the injection flow from a syringe to a patient.

In some embodiments, such as seen in FIG. 5A and FIG. 5B, the inflow 502 and outflow 504 fluid passages may be in line with each other, and the twist cap 506 may be oriented perpendicularly to the fluid pathways. In some embodiments, such as seen in FIG. 5C, the inflow 502 and outflow 504 fluid passages may be oriented perpendicular to each other.

In some embodiments, twisting the cap 506 adjusts a width of a valve 508. The aperture size of the valve width 508 may range from fully open, such as seen in FIG. 5D, partially closed, such as seen in FIG. 5E, and fully closed, such as seen in FIG. 5F. Twisting the cap 506 may twist a pathway such that the pathway is fully aligned with the valve 508. When the pathway controlled by the twisting cap 506 is fully aligned with the valve 508, the valve may be completely open. Twisting the cap 506 such that the pathway is offset from the valve 508 may allow the valve 508 to be partially or completely closed, thus controlling the fluid flow through the valve 508 relative to how open or closed the valve 508 is.

The disclosures described herein include at least the following clauses:

Clause 1: A device for rapid fluid delivery, the device comprising: a pre-filled syringe for providing an infusion, the syringe comprising: a syringe barrel for holding a fluid; and a syringe plunger connected to a proximal end of the barrel; and a rapid infusion attachment, wherein the rapid infusion attachment provides increased pressure to drive an infusion, the rapid infusion attachment comprising: an attachment plunger connected to the syringe plunger by a spring; a housing that sheaths a portion of the attachment plunger; and an activation mechanism that controls flow of the fluid from the syringe.

The device of Clause 1, wherein the attachment plunger comprises a base portion and a hollow channel portion, wherein the hollow channel portion houses the spring.

The device of Clause 2, wherein the attachment plunger slides to sheath the hollow channel portion within the housing.

The device of Clause 3, wherein the spring is attached to a proximal end of the syringe plunger and the base portion of the hollow channel of the attachment plunger, and wherein when the hollow channel portion is sheathed within the housing, the spring is in a compressed position, and wherein the spring provides an injection force to the syringe plunger when the spring is in the compressed position.

The device of Clause 4, wherein the hollow channel portion is sheathed within the housing, compressing the spring, before being packaged.

The device of Clause 4, wherein the hollow channel portion of the attachment plunger comprises exterior threads and the housing comprises interior threads that interact with the exterior threads of the hollow channel portion, and wherein the attachment plunger twists to sheath the hollow channel portion within the housing.

The device of Clause 4, wherein the attachment plunger comprises a protruding flange at a distal end of the hollow channel portion and wherein the housing comprises interior grooves that catch the protruding flange of the attachment plunger such that when a compression force is applied to a proximal end of the attachment plunger, the hollow channel portion is sheathed within the housing and the protruding flange interacts with the interior grooves to keep the attachment plunger in its sheathed position.

The device of Clause 1, wherein the activation mechanism is attached to a needle end of the syringe and wherein the activation mechanism stops the fluid from flowing out of the syringe when closed and allows the fluid to flow out of the syringe when open.

The device of Clause 1, wherein a distal end of the syringe facilitates attachment of the device to a venous access pathway.

The device of Clause 1, wherein the syringe barrel is pre-filled with sterile saline or other fluid for a rapid fluid injection.

The device of Clause 1, wherein the attachment housing removably clips to the pre-filled syringe.

A device for rapid fluid delivery, the device comprising: a pre-filled syringe for providing an infusion, the syringe comprising: a syringe barrel with a proximal end and a distal end, wherein the barrel is filled with a fluid; and a syringe plunger with a gasket end and a free end, wherein the gasket end is located within the barrel; and a device plunger with a base portion and an elongate core portion, wherein the elongate core portion comprises a hollow chamber configured to receive the syringe plunger; a housing with a proximal end and a distal end, wherein the distal end of the housing is attached to the proximal end of the syringe barrel, wherein the housing is configured to receive the elongate core portion of the device plunger; and a pre-compressed spring attached to the free end of the syringe plunger and to the device plunger, wherein the pre-compressed spring provides an injection force to the syringe plunger.

The device of Clause 12, further comprising a flow regulating device attached to a tip of the syringe barrel, wherein the flow regulating device controls fluid flow from the syringe barrel.

The device of Clause 13, wherein the flow regulating device comprises a valve mechanism configured to open and close a fluid pathway.

The device of Clause 14, wherein opening the fluid pathway permits the fluid to be pushed out of the syringe barrel as the spring decompresses.

The device of Clause 14, wherein the valve mechanism is electronic and programmable such that the valve mechanism opens and closes the fluid pathway on a timer to administer the fluid at a predetermined rate.

The device of Clause 12, wherein the pre-filled syringe is configured to be inserted into an IV system or a patient.

A device for automatic syringe dispensing, the device comprising: a syringe barrel having a tip end and a base end; a spring located within the syringe barrel and attached to the base end of the syringe barrel; a locking mechanism located within the syringe barrel and adjacent to the spring; and a plunger located within the syringe barrel and adjacent to the locking mechanism; wherein the locking mechanism prohibits movement of the plunger when active and permits movement of the plunger when released.

The device of Clause 18, wherein the syringe barrel is filled with a fluid, and wherein the fluid is pushed out of the syringe barrel by the spring when the locking mechanism is released.

The device of Clause 19, further comprising a flow regulator attached to the tip end of the syringe, wherein the flow regulator is configurable to infuse the fluid at a determined rate.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled”or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S. C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S. C. § 101, 102, or 103, nor should they be interpreted in such a way.

What is claimed is:

1. A device for rapid fluid delivery, the device comprising:
a pre-filled syringe for providing an infusion, the syringe comprising:
a syringe barrel for holding a fluid; and
a syringe plunger connected to a proximal end of the barrel; and
a rapid infusion attachment, wherein the rapid infusion attachment provides increased pressure to drive an infusion, the rapid infusion attachment comprising:
an attachment plunger connected to the syringe plunger by a spring;
a housing that sheaths a portion of the attachment plunger; and
an activation mechanism that controls flow of the fluid from the syringe.

2. The device of claim 1, wherein the attachment plunger comprises a base portion and a hollow channel portion, wherein the hollow channel portion houses the spring.

3. The device of claim 2, wherein the attachment plunger slides to sheath the hollow channel portion within the housing.

4. The device of claim 3, wherein the spring is attached to a proximal end of the syringe plunger and the base portion of the hollow channel of the attachment plunger, and wherein when the hollow channel portion is sheathed within the housing, the spring is in a compressed position, and wherein the spring provides an injection force to the syringe plunger when the spring is in the compressed position.

5. The device of claim 4, wherein the hollow channel portion is sheathed within the housing, compressing the spring, before being packaged.

6. The device of claim 4, wherein the hollow channel portion of the attachment plunger comprises exterior threads and the housing comprises interior threads that interact with the exterior threads of the hollow channel portion, and wherein the attachment plunger twists to sheath the hollow channel portion within the housing.

7. The device of claim 4, wherein the attachment plunger comprises a protruding flange at a distal end of the hollow channel portion and wherein the housing comprises interior grooves that catch the protruding flange of the attachment plunger such that when a compression force is applied to a proximal end of the attachment plunger, the hollow channel portion is sheathed within the housing and the protruding flange interacts with the interior grooves to keep the attachment plunger in its sheathed position.

8. The device of claim 1, wherein the activation mechanism is attached to a needle end of the syringe and wherein the activation mechanism stops the fluid from flowing out of the syringe when closed and allows the fluid to flow out of the syringe when open.

9. The device of claim 1, wherein a distal end of the syringe facilitates attachment of the device to a venous access pathway.

10. The device of claim 1, wherein the syringe barrel is pre-filled with sterile saline or other fluid for a rapid fluid injection.

11. The device of claim 1, wherein the housing removably clips to the pre-filled syringe.

12. A device for rapid fluid delivery, the device comprising:
a pre-filled syringe for providing an infusion, the syringe comprising:
a syringe barrel with a proximal end and a distal end, wherein the barrel is filled with a fluid; and
a syringe plunger with a gasket end and a free end, wherein the gasket end is located within the barrel; and
a device plunger with a base portion and an elongate core portion, wherein the elongate core portion comprises a hollow chamber configured to receive the syringe plunger;
a housing with a proximal end and a distal end, wherein the distal end of the housing is attached to the proximal end of the syringe barrel, wherein the housing is configured to receive the elongate core portion of the device plunger; and
a pre-compressed spring attached to the free end of the syringe plunger and to the device plunger, wherein the pre-compressed spring provides an injection force to the syringe plunger.

13. The device of claim 12, further comprising a flow regulating device attached to a tip of the syringe barrel, wherein the flow regulating device controls fluid flow from the syringe barrel.

14. The device of claim 13, wherein the flow regulating device comprises a valve mechanism configured to open and close a fluid pathway.

15. The device of claim 14, wherein opening the fluid pathway permits the fluid to be pushed out of the syringe barrel as the spring decompresses.

16. The device of claim 14, wherein the valve mechanism is electronic and programmable such that the valve mechanism opens and closes the fluid pathway on a timer to administer the fluid at a predetermined rate.

17. The device of claim 12, wherein the pre-filled syringe is configured to be inserted into an IV system or a patient.

18. A device for automatic syringe dispensing, the device comprising:
a syringe barrel having a tip end and a base end;
a spring located within the syringe barrel and attached to the base end of the syringe barrel;
a locking mechanism located within the syringe barrel and adjacent to the spring; and
a plunger located within the syringe barrel and adjacent to the locking mechanism;
wherein the locking mechanism prohibits movement of the plunger when active and permits movement of the plunger when released.

19. The device of claim 18, wherein the syringe barrel is filled with a fluid, and wherein the fluid is pushed out of the syringe barrel by the spring when the locking mechanism is released.

20. The device of claim 19, further comprising a flow regulator attached to the tip end of the syringe, wherein the flow regulator is configurable to infuse the fluid at a determined rate.
